(19) United States
(12) Patent Application Publication
(10) Pub. No.: US 2010/0034838 A1
(43) Pub. Date: Feb. 11, 2010

(54) Transdermal Administration of Active Agents for Systemic Effect

(76) Inventors: John Staniforth, Wiltshire (GB); Paul Goggin, Wiltshire (GB)

Correspondence Address: Davidson, Davidson & Kappel, LLC, 485 7th Avenue, 14th Floor, New York, NY, 10018, US

(21) Appl. No.: 12/086,047

(22) PCT Filed: Dec. 7, 2006
(86) PCT No.: PCT/GB2006/050437

§ 371 Date: Sep. 18, 2009

(30) Foreign Application Priority Data
Dec. 7, 2005 (GB) 0524961.0

(51) Int. Cl. A61K 31/57; A61K 39/00; A61P 1/08; A61P 25/16; A61P 25/34; A61K 47/14; A61K 47/46; A61K 47/12; A61K 47/44; A61K 31/4439; A61K 31/137; A61K 31/568; A61K 31/565

(52) U.S. Cl. 424/184.1; 514/786; 514/783; 514/784; 514/343; 514/649; 514/178; 514/177; 514/182

(57) ABSTRACT

The present invention relates to compositions for transdermal administration of a therapeutic agent for providing a systemic therapeutic effect. In particular, the invention relates to spreadable compositions, or compositions which may be solid at a temperature of about 25° C. or less and have a softening point of not higher than 35° C., wherein transdermal administration of the therapeutic agent may be either rapid or sustained.

The present invention relates to compositions for transdermal administration of therapeutic agents for providing a systemic therapeutic effect.

Transdermal absorption is a recognized means of systemic drug administration which benefits from being a non-invasive and convenient way of medicating a patient. Transdermal absorption is a potentially useful means of drug administration for patients who find other methods difficult or unpleasant. For example, the young and the old can have difficulty with orally administered medications and may find injections particularly unpleasant. Children and patients with dementia can also be difficult to medicate due to lack of compliance. As such, transdermal administration of therapeutic agents could be a valuable method of administering a medication, especially in the young, the old or mentally impaired patients.

However, systemic delivery of drugs across the skin currently has limitations. The outer most layer of the skin, the stratum corneum, is composed of dead keratin-rich cells (corneocytes) and a lipid matrix. The stratum corneum is 10-15 μm thick in adults and forms an effective barrier membrane that limits the type of molecules that can be absorbed by the skin, and also the rate of absorption. As a result, transdermal administration of compositions containing therapeutic agents typically provides slow, sustained administration of the therapeutic agent through the skin over a period of time. In order to ensure that the compositions remain in contact with the skin for this gradual transdermal absorption, compositions are usually incorporated into a patch or plaster which is adhered to the skin.

Such “medicinal plasters” are well known in the art. For example, hormone-containing patches, such as Estraderm® or Climara®, are used in hormone replacement therapies (HRT) and nicotine-containing patches can be used as part of a nicotine-replacement program by people who are giving up smoking. Medicinal plasters have several advantages over other means of providing sustained levels of a drug in a patient. For example, medicinal plasters avoid the need for frequent dosing in order to achieve sustained drug effects. Medicinal plasters also provide an easy and non-invasive way of administering a drug.

There are currently three major types of transdermal patch systems, namely membrane-controlled systems, adhesive diffusion-controlled systems or matrix systems.

The membrane-controlled system typically consists of four layers: an impermeable backing layer, a polymer layer that serves as a drug reservoir, a rate-controlling microporous membrane, and an adhesive. The drug reservoir comprises the therapeutic agent and liquid excipients that encourage absorption of the drug across the skin. Upon application to the skin, the drug diffuses through the membrane and then passes through the adhesive before reaching the skin. The drug release rate is constant, so, in order to provide the most efficient method of administration, the release rate must be maintained at a level just below the saturation limit of the skin. U.S. Pat. No. 5,683,712 discloses an example of a membrane-controlled system for transdermal administration of homeopathic drugs, wherein a micro-porous membrane is provided for controlling the release of the drug, with a gel containing the drug scattered within the membrane.

The adhesive diffusion-controlled system is very similar to the membrane-controlled system except that the rate-controlling microporous membrane is absent. The system consists instead of an impermeable plastic barrier, a drug reservoir and one or more rate controlling adhesive layers next to the skin. DE 19849823 discloses an example if an adhesive diffusion-controlled system, the medicinal plaster comprising a backing layer, a reservoir containing the active agent and an adhesive layer overlying the reservoir layer which has non-adhesive regions allowing passage of the active agent on contact with the skin.

In the matrix system, the drug reservoir is in direct contact with the skin. As such, the rate of diffusion of the drug is dependent upon the absorption rate of the skin. The system may comprise an impermeable backing attached to a drug reservoir consisting of a hydrophilic or hydrophobic polymer containing the dispersed drug. WO 87/00042 describes such a system, for transdermal administration of verapamil at a sustained, substantially uniform rate over an extended period of time. Alternatively, the system may comprise a backing, and an adhesive layer which serves a dual purpose as both the adhesive and the drug reservoir. An example of such a system is described in WO 00/02539, which relates to a plaster containing a non-steroidal anti-rheumatic agent.

However, the known plaster systems suffer from several disadvantages. Each system entails release of the drug from a material reservoir, which is, typically, a tissue tolerant polymer. As such, the surface area for absorption of the drug is, in each case, limited to the surface area of the material that is in contact with the skin. The production of membrane, matrix and adhesion-controlled systems is also technically costly and requires special apparatus.

Further disadvantages are associated with the use of adhesive plasters for transdermal administration of therapeutic agents. For example, the adhesive used on plasters has to be compatible with the therapeutic agent and other constituents of the composition used with the plaster. This means that such plasters can be expensive and that not all therapeutic agents are suitable for inclusion in them. Additionally, the application of a plaster can be both impractical and undesirable, especially if the plaster is attached to the skin for a prolonged period of time.

An object of the present invention is therefore to provide a composition comprising one or more therapeutic agents and a pharmaceutically acceptable carrier, which provides a relatively simple and inexpensive yet effective means of sustained systemic transdermal administration of the therapeutic agent.

The term “sustained” as used herein relates to the provision of a composition from which a therapeutic agent can be absorbed by the skin over a period of time, for example, over a period of at least about 30 minutes, or at least about 1, 2, 4, 8, 12, 16, 20, 24, 26, 28, 32, 36, 48 or 72 hours.

A further object of the present invention is to provide compositions for sustained systemic transdermal administration of one or more therapeutic agents that do not need to be incorporated into a plaster or patch, thereby avoiding the need for the compositions to be provided with a covering which has to be affixed to the skin using an adhesive.

The term “therapeutic agent”, “active agent” or “pharmaceutically active agent” denotes any pharmaceutically active substance suitable for topical application and transdermal administration to a patient (particularly a human patient). It is preferred that the therapeutic agent is a systemically active agent that is absorbed through the skin, which provides a systemic therapeutic effect. However, some therapeutic agents may also have a local effect following absorption through the skin. Such agents include all of the drugs and classes of drugs referred to in the following passages, plus pharmaceutically acceptable equivalents thereof, such as their pharmaceutically acceptable salts, esters, prodrugs and active metabolites. Isomers of all disclosed agents are also encompassed by this disclosure.

The term “systemic administration” as used herein relates to the administration of the therapeutic agent to the blood stream.

The term “local administration” as used herein relates to the provision of a composition containing one or more therapeutic agents for application to the skin of a patient, wherein the therapeutic agent has an effect on the area of skin to which it is applied, upon receptors in the skin, and/or upon receptors in the layers of the skin within close proximity to the site of application of the composition, and wherein the therapeutic agent is not administered to the bloodstream.

Many medical conditions require or benefit from rapid administration of a therapeutic agent into the bloodstream. For example, in situations where rapid alleviation of symptoms is desirable, such as in nausea or vomiting, or for the treatment of any symptom in a neonate, infant or young child, who can become very distressed when ill. Rapid symptomatic relief is also highly desirable in patients suffering from incapacitating symptoms such as those associated with, for example, Parkinson's disease.

The most common route of administering pharmaceutically active agents is probably the oral route. However, this route of administration has drawbacks. Tablets frequently have a delayed onset of action. As such, tablets are unsuitable in situations where rapid relief of symptoms is required. Further to this, tablets can be difficult for certain patients to swallow and can also present a risk of choking in some patients, for example, the old or the young. Also, in some circumstances it is undesirable or difficult to swallow a tablet, for example, when suffering from nausea and/or vomiting.

Administering therapeutic agents by injection is a known way of providing a rapid onset of the therapeutic effect. However, this is an inconvenient and impractical mode of drug administration due to the need for equipment such as a needle and syringe, and the frequent requirement that a medically trained person administers the injection. Injection is also an invasive and unpleasant mode of administration, which is particularly undesirable in neonates, infants, young children and the elderly.

Other modes of rapid drug administration include inhalers or nasal sprays. Disadvantages of such methods of administration include that they are invasive, a bulky device is required in order to administer the drug, and many people find nasal sprays and inhalers difficult or unpleasant to use.

Clearly, there is a need for an easy, non-invasive mode of rapid drug administration. Transdermal absorption, as hereinbefore discussed, satisfies both of these criteria. However, until now transdermal absorption has not been considered to be a feasible method of rapid drug administration due to the limitations on absorption rate imposed by the stratum corneum, as discussed above.

Thus, a further object of the present invention is to provide a composition comprising one or more therapeutic agents, wherein the composition is suitable for rapid systemic transdermal administration.

The term “rapid” as used herein relates to the absorption of a therapeutic agent into the bloodstream within, for example, less than about 20, 15, 10, 5, 4, 3 or 2 minutes, or less than about 1 minute or 0.5 minutes from application to the skin.

In particular, an object of the present invention is to provide a composition which can simply be spread over an area of skin, from where it is rapidly absorbed, resulting in quick delivery of the therapeutic agent to the bloodstream, thereby providing a rapid therapeutic effect. Such a composition would be of enormous benefit in situations where rapid alleviation of symptoms is desirable, and where it is undesirable for the patient to have to use an invasive means of drug administration.

The compositions provided by the present invention achieve the object of rapid or sustained transdermal absorption by the use of appropriate compositions which control the release of the active agent when applied to the skin, and therefore the delivery kinetics. This means that the rate of delivery of an active agent to the skin from a composition according to the present invention can be altered in accordance with therapeutic requirements. The rate of delivery of the active agent to the skin can be modified by altering the affinity of the active agent for the carrier material compared to the affinity of the active agent for transportation through the stratum corneum. This is achieved by the preparation and use of particular carrier materials in the composition, which are tailored to the active agents in question, in order to alter the hydrophilicity of the composition. Using this approach, the solvent properties of the composition, and therefore the solubility profile of the active agent can be modified, which means that the rate of diffusion of the active agent can be controlled.

According to a first aspect of the present invention, there is provided a pharmaceutical composition comprising a therapeutic agent and a pharmaceutically acceptable carrier, the composition being suitable for topical application to the skin of a mammalian patient resulting in systemic transdermal administration of the therapeutic agent, thereby providing a therapeutically effective plasma concentration of the therapeutic agent within a period of no more than about 20 minutes from application to the skin.

In certain embodiments, the composition provides a therapeutically effective plasma concentration of the therapeutic agent within a period of less than 15, 10, 5, 4, 3 or 2 minutes, or less than 1 minute or 0.5 minutes from application to the skin.

The pharmaceutical compositions according to the first aspect of the present invention preferably further comprise a means of substantially occluding the therapeutic agent from the air following application to the skin. The occlusive means is provided by the use of appropriate quantities of wax, oil or fat included in the carrier material of the composition.

Providing pharmaceutical compositions comprising a means of occlusion, which are suitable for transdermal administration, serves several purposes. An important factor in the rate of absorption of a substance through the skin is the hydration level of the skin. Application of an occlusive layer to the skin causes a local increase in temperature, causing dilatation of the pores in the skin and sweating, thereby hydrating the skin and enhancing absorption of agents that have been applied to the skin, preferably located between the occlusive layer and the skin.

The pharmaceutical compositions for rapid administration of a therapeutic agent according to the present invention are for use in therapy or prophylaxis. The conditions to be treated include, for example, nausea and vomiting, Parkinson's disease or essential tremor, chorea, tics and related disorders. The compositions may also be used in nicotine-replacement therapy, the compositions being able to provide a rapid nicotine “hit”, which simulates smoking a cigarette. The compositions may also be used in the administration vaccines. In preferred embodiments, the compositions for rapid administration of a therapeutic agent according to the present invention are not used for the treatment of respiratory diseases, pain or inflammation, or the administration of local anesthetics.

Therapeutic agents which may advantageously be included in the compositions of the present invention for rapid administration include those which are usually administered for the treatment of: pain and/or inflammation, for example, ibuprofen, aceclofenac, acemetacin, azapropazone, celecoxib, dexketoprofen, diclofenac, diflunisal, etodolac, etoricoxib, fenbufen, fenoprofen, flurbiprofen, indometacin, ketoprofen, mefenamic acid, meloxicam, nabumetone, naproxen, piroxicam, rofecoxib, sulindac, tenoxicam, tiaprofenic acid, valdecoxib, glyceryl trinitrate, opioids such as fentanyl, buprenorphine, salicylic acid and related salicylates; nausea and/or vomiting, for example, hyoscine hydrobromide, dolasetron, granisetron, ondansetron, tropisetron, scopolamine, 5HT₃ receptor antagonists; Parkinson's disease, for example, levodopa, co-beneldopa, co-careldopa, amantadine hydrochloride, apomorphine hydrochloride, bromocriptine, cabergoline, entacapone, lisuride maleate, pergolide, pramipexole, ropinirole, selegeline hydrochloride or antimuscarinic agents, for example, benzatropine mesilate, orphenadrine hydrochloride, procyclidine hydrochloride, trihexyphenidyl hydrochloride; essential tremor, chorea, tics and related disorders, for example, haloperidol, piracetam, riluzole, tetrabenazine, botulinum toxin A and B. The therapeutic agent may be one used for assisting smoking cessation, for example, nicotine or bupropion hydrochloride. Alternatively, the agent may be one known for use in vaccines, for example for vaccinating against anthrax, cholera, diphtheria, haemophilus influenzae type B, hepatitis A or B, influenza, measles, MMR (measles, mumps and rubella), meningococcal, mumps, pertussis, pneumococcal, poliomyelitis, rabies, rubella, smallpox, tetanus, tick-borne encephalitis, typhoid, varicella-zoster, yellow fever. The therapeutic agent may be botulism antitoxin or bacillus of calmette and guerin (BCG). Alternatively, the therapeutic agent may be a nitrate, and in particular, a fast onset nitrate, for the treatment of angina and/or related conditions, such as glyceryl trinitrate or isosorbide mono- or dinitrate.

Pharmaceutical compositions according to the present invention may comprise more than one therapeutic agent, provided that they are compatible with one another under conditions of storage and use. One possible combination is an NSAID and an opioid, for example, diclofenac and fentanyl.

In some embodiments of the present invention, the compositions intended for rapid transdermal administration are spreadable. For example, these compositions are provided in the form of a cream, ointment or gel. Such spreadable compositions have the advantage that they are easily applied and rubbed into the skin in order to aid absorption.

In embodiments of the present invention where all of the therapeutic agent in the composition is to be administered as quickly as possible, the carrier medium allows substantially complete absorption of the therapeutic agent through the skin of the mammalian patient, so as to effect what is preferably substantially complete administration of the therapeutic agent to the mammalian patient, within a time period of less than about 20 minutes, less than about 15 minutes, less than about 10 minutes, preferably less than about 5 minutes, more preferably less than about 3 minutes and most preferably less than about 1 minute following application of the composition to an area of skin.

In embodiments of the present invention where the pharmaceutical composition is provided as a spreadable composition for rapid administration, the carrier medium should allow the therapeutic agent to be carried in a stable manner. The carrier medium may have favourable organoleptic properties, for example, the composition may be water-based so as to have a non-oily feel upon application to the skin. A carrier medium should be compatible with the therapeutic agent, affording the therapeutic agent chemical stability. The make-up of the compositions of the present invention should be designed to encourage the flux of drug from the composition and through the stratum corneum.

Components which are commonly used as a base for creams, ointments or gels may be used as a carrier medium in the compositions according to the present invention. These components include: water; hydrocarbon oils and waxes; silicone oils; vegetable, animal or marine fats or oils; glycerides (such as, for example, or more glycerol esters of saturated fatty acids or polyglycolysed glycerides, cocoa butter, theobroma or the like) or glyceride derivatives; high molecular weight polyethylene glycol, polyoxyethylene, lanolin and derivatives thereof; fatty acids, fatty alcohols or fatty esters (including, for example, caprylic acid, caprylic triglyceride or the like); lecithin; polyhydric alcohols or esters; wax esters; sterols; phospholipids and the like. Thickening agents such as gums or other forms of hydrophilic colloids may be included. The carrier medium may comprise more than one base component.

Where the pharmaceutical composition is a cream, the carrier medium may comprise substantially more oil based components than water. Where the pharmaceutical composition is an ointment, the carrier medium may comprise substantially more water than oil based components. Where the pharmaceutical composition is a gel, the carrier medium may substantially comprise water.

Where the composition is provided as a cream, ointment or gel, it is possible to accurately control the dose to be applied to the skin of the patient by providing one or more unit or measured doses of the spreadable composition. This helps to ensure that the patient receives an accurate, predetermined dose of therapeutic agent. In the present invention, such unit or measured doses may be packaged individually, for exampled in containers such as tubes or sachets. Alternatively, the compositions of the present invention may be dispensed by a device which is capable of dispensing an accurate, predetermined amount.

In alternative embodiments of the present invention, compositions for rapid transdermal administration have a substantially solid form at a temperature of about 25° C. or less, and a softening point of not higher than the skin temperature of a mammalian patient as substantially hereinafter described. More particularly, the pharmaceutical composition is in a substantially solid form during storage (at a temperature of or below 25° C.). However, following application of the composition to an area of the skin of a mammalian patient, the solid composition softens to a consistency that can be substantially absorbed by the area of skin so as to effect transdermal administration of the therapeutic agent to the mammalian patient.

Compositions of the present invention should be stored at temperatures of about 25° C. or less, in accordance with storage conditions for most pharmaceutical compositions and formulations.

The provision of compositions for rapid transdermal administration which are solid at temperatures of about 25° C. or less allows ease of handling and application of the composition. Softening of the composition of the present invention upon contact with the skin, however, allows the composition to enjoy the favourable absorption properties and ease of spreading associated with non-solid compositions. Spreading of the softened composition upon contact with the skin also facilitates the passage of the active agent from the composition through the stratum corneum, by increasing the area of the composition in contact with the skin, thereby providing a greater surface area for the active agent to diffuse through the stratum corneum in accordance with the classical diffusion theory.

The carrier medium used in the compositions of the present invention which soften upon contact with skin is selected to be substantially solid at a temperature of about 25° C. or less, and to soften to a consistency that allows for substantially complete absorption of the one or more therapeutic agents by the area of skin of the mammalian patient at a temperature of above 25° C. It is preferred that the carrier medium softens, and advantageously may be converted to a spreading consistency, at a temperature in the range of 30 to 35° C. or up to around 37° C.

The compositions of the present invention are preferably provided for application to a human patient. In such embodiments, the compositions preferably have a softening point which is not higher than the normal temperature at the skin surface (skin temperature) of a human. This temperature is typically not higher than about 35° C. In certain embodiments, the composition has a softening point from about 25° C. to about 35° C., or from about 30° C. to not higher than about 35° C.

In one embodiment of the invention, the pharmaceutical composition is solid at a temperature of about 25° C. or less and has a softening point of not higher than 35° C., such that when the composition is placed in continuous contact with the skin of a mammalian patient, it is softened to a consistency to effect substantial application of the therapeutic agent onto a desired skin area of the mammalian patient within a time period of less than 10 minutes.

This allows for substantially complete absorption of the composition over the area of skin, so as to effect substantially complete administration of the therapeutic agent to the mammalian patient.

The term “softening point” as used herein refers to a temperature at which a substantially solid dosage form starts to soften to a consistency that can be absorbed by the skin of a patient, so as to allow transdermal absorption of the therapeutic agent present in the composition.

The softening point of a substantially solid dosage form of a pharmaceutical composition according to the first aspect of the present invention can be determined visibly as the temperature at which the substantially solid dosage form starts to soften to a consistency that can be spread and absorbed by the skin of a patient and as such can advantageously be substantially completely absorbed by the skin of the patient so as to leave little or no undesirable residue on the skin of a patient.

Alternatively, the softening point of a substantially solid dosage form of a pharmaceutical composition according to the first aspect of the present invention can be determined using a TA-XT2 texture analyser (Stable MicroSystems Ltd., UK), suitably equipped with a 5 kg load cell. The equipment is enclosed in a temperature controlled chamber (capable of operating in the region of 60° C. to 200° C.). A tablet or other substantially solid dosage form according to the present invention may be enclosed in the chamber at the specified temperature for a time of at least 10 minutes. A 3 mm flat faced probe is pushed into the tablet or other substantially solid dosage form according to the present invention for a distance of 1 mm at a speed of 0.1 mm/sec. Measurements can be repeated at temperature increments of 1° C. and, at the temperature at which the peak force of resistance recorded (as measured by Texture Exceed software) falls to below 50% of that for a “solid” tablet or other substantially solid dosage form according to the present invention, the tablet or other dosage form is deemed to have “softened”.

The term “spreading point” as used herein refers to a temperature at which the composition has a spreading consistency. For example, the composition may flow under its own weight or at least can be spread upon the skin of a mammalian patient, for example, using finger pressure.

The mobility of a spreading composition may promote the absorption of the therapeutic agent into the skin by allowing movement of the therapeutic agent towards the skin, for example, by diffusion. The spreading point of a preparation may be measured using the TA-XT2 texture analyser mentioned above in relation to measurement of softening point and with this analyser the spreading point of a composition is the temperature at which outward flow of the composition is first observed on advance of the flat faced probe into the preparation.

In another embodiment, the pharmaceutical composition suitable for topical administration, preferably to a mammal, comprises one or more therapeutic agents and a carrier medium, wherein said preparation has a softening point of not higher than skin temperature of a mammalian patient, said composition having an aspect ratio (wall:face) of less than 1:1.

In another embodiment, the pharmaceutical composition for topical administration, preferably to a mammal, comprises a compacted granulate including one or more therapeutic agents and a pharmaceutically acceptable carrier, said compacted granulate having a softening point of not higher than skin temperature of the intended subject, preferably a mammalian patient.

In certain embodiments, the composition, which is solid prior to administration by application to the skin, has a shape to facilitate the topical application. For example, the composition can have: at least one flat surface; at least one concave surface; at least one convex surface; two flat surfaces; two concave surfaces; or two convex surfaces. The composition may be in the form of a standard tablet, spherical or half-spherical. Bullet shaped and conical shaped compositions are not preferred in the present invention.

In preferred embodiments, the compositions of the present invention have a total weight of from about 50 mg to less than 1 g, preferably from about 100 mg to about 900 mg and more preferably from about 250 mg to about 750 mg. The compositions of the present invention can have a total weight of 1 g or greater, if desired.

In certain embodiments of the present invention, the compositions are provided as a solid unit dosage form and comprise a therapeutically effective amount of at least one therapeutic agent for topical application to a mammalian patient. The unit dosage form is a solid during final manufacture and has, upon application to an area of skin of said mammalian patient, a spreading consistency suitable for application to said area of skin. Unit dosage forms may be packaged individually, for example, in a plastic container having a removable or breakable enclosure for dispensing said unit dosage form.

Typically, the substantially solid unit dosage form is provided in the form of a tablet or of a rolled preparation, for example, a pill or the like.

In certain embodiments, the dosage form can be a plurality of substantially discrete substantially solid particles comprising one or more therapeutic agents admixed with a pharmaceutically acceptable carrier, said particles having a softening point of about 30° C. to about 35° C. The particles can be enclosed in a sachet, a capsule or a device suitable to dispense an individual dose of the particles.

More particularly, it is preferred that the shape and configuration of the substantially solid dosage form is determined by the softening point of the composition and/or the carrier medium. It may be preferred that a substantially solid dosage form according to the present invention comprises a substantially unitary form; alternatively, it may comprise a plurality of discrete particles (such as a plurality of granules or the like) that can be absorbed by the skin of a mammalian patient. Preferably, the plurality of substantially discrete particles are provided in a sealed member (such as a capsule, sachet, blister package or the like) from which they are dispensed and applied to the skin of a patient.

Any component commonly used for suppositories can be used as carriers in the compositions of the present invention which soften upon application to the skin. These components include those derived from mammalian, vegetable or mineral origins, and materials partially or totally synthesized. Specific examples of such carriers include oils and fats of mammalians or vegetable origin, such as olive oil, corn oil, castor oil, cottonseed oil, wheat germ oil, cacao butter, hydrogenated oils, etc.; hydrocarbons, such as squalane, petrolatum, solid paraffin, liquid paraffin, etc.; and waxes, such as jojoba oil, carnauba wax, bees wax, lanolin, etc. Examples of partially or totally synthesized fatty acid esters include glycerol, mono-, di-, or triglycerides of medium or higher fatty acid, such as saturated linear fatty acid, for example lauric acid, myristic acid, palmitic acid, stearic acid, etc., or unsaturated linear fatty acids, for example oleic acid, linoleic acid, linolenic acid, etc. Commercially available carriers which are suitable include Witepsol (manufactured by Dynamit Nobel), Pharmasol (manufactured by Nippon Oil and Fats Co.), Isocacao (manufactured by Kao Corp.), SB (manufactured by Taiyo Oil and Fats Co.), Novata (manufactured by Henkel), Suppocire (manufactured by Gattefosse Co.), and the like. Examples of other synthetic products include polyethylene glycol, for example, macrogole, setomacrogole, etc., as well as derivatives thereof, for example, setomacrogol.

In order to obtain the desired softening point of the compositions of the present invention, different carriers can, if necessary, be combined in order to increase or decrease the softening point to obtain a suitable product. For example, in order to decrease the softening point, a plasticizer can be added, e.g., glyceryl monostearate, myristyl alcohol, polysorbate 80, propylene glycol or combinations thereof. In order to increase the softening point, a hardener can be added, e.g., beeswax, cetyl alcohol, stearic acid, stearyl alcohol, aluminium monostearate, aluminium distearate, aluminium tristearate, bentonite, magnesium stearate, colloidal silicon dioxide or combinations thereof.

A carrier for use according to the present invention may comprise any ingredient suitable for use in a pharmaceutical composition and possessing the desired properties for enabling topical administration of a dose of at least one therapeutic agent, provided that it is suitable for topical application and transdermal administration. For example, the carrier may include a cellulose or one or more ingredients selected from the group consisting of ingredients of the type suitable for use in suppositories including, for example, one or more glycerides (such as, for example, one or more glycerol esters of saturated fatty acids or one or more polyglycolysed glycerides, cocoa butter, theobroma or the like), one or more high molecular weight polyethylene glycol, one or more polyoxyethylene, lanolin and derivatives thereof, and one or more fatty acids, fatty alcohols, fatty acid esters (including, for example, caprylic acid, caprylic triglyceride or the like), and any of the preceding ingredients can be optionally mixed with one or more organic oils (including, for example hydrogenated vegetable oils) or the like.

It is often preferred that a carrier employed in a pharmaceutical composition according to the present invention comprises, and more preferably consists essentially of, one or more glycerides, including, in particular, one or more glycerol esters of C8-C18 fatty acids or one or more polyglycolysed glycerides.

Suitably, the carrier of a pharmaceutical composition according to the present invention comprises, or consists essentially of, a mixture of glycerides, where the glycerides can be one or more mono-, di- or tri-glycerides, optionally wherein the glycerides comprise glycerol esters of C12-C18 fatty acids. In one embodiment, the glyceride mixture is a Witepsol grade product. More particularly, the carrier may comprise, or consist essentially of, a Witepsol grade product available under any of the trade marks Witepsol H5, Witepsol H15, Witepsol H32, Witepsol S51, Witepsol S55, Witepsol S58, Witepsol W25 and Witepsol W32. In a particularly preferred embodiment, the pharmaceutical compositions according to the present invention include carriers which are Witepsol grade products available under any of the following trade marks Witepsol H5, Witepsol H15, Witepsol S51 and Witepsol S55. The Witepsol grade product available under the trade mark Witepsol H15 is particularly suitable.

In a particular embodiment of the present invention, the carrier employed in the compositions consists essentially of a Witepsol grade product substantially as described above.

Alternatively, the carrier comprises, or consists essentially of, a mixture of glycerides, where the glycerides can be selected from the group consisting of mono-, di- and tri-glycerides, the glycerides comprising glycerol esters of Cg-Cie fatty acids or one or more polyglycolysed glycerides. In one embodiment, glyceride mixtures available under the trade marks Gelucire or Suppocire are used, such as any of the following: Gelucire 33/01, Gelucire 39/01, Gelucire 43/01, Gelucire 44/14, or any of the Suppocire Standard type, Suppocire N type or Suppocire P type products.

Alternatively, the carrier used in a pharmaceutical composition according to the present invention comprises, or consists essentially of, cocoa butter.

Methods of preparing the softening compositions referred to above are disclosed in WO 02/00203 A1.

The technology disclosed herein can also be applied to pharmaceutical compositions for providing sustained transdermal administration of the therapeutic agent.

According to a second aspect of the present invention, there is provided a pharmaceutical composition comprising a therapeutic agent and a pharmaceutically acceptable carrier, the composition being suitable for topical application to the skin of a mammalian patient resulting in systemic transdermal administration of the therapeutic agent over a period greater than 30 minutes from application to the skin.

In certain embodiments, the composition provides transdermal administration of the therapeutic agent over a period greater than about 1, 2, 4, 8, 12, 16, 20, 24, 26, 28, 32, 36, 48 or 72 hours from application to the skin. The onset of the therapeutic effect may, in some embodiments, also be rapid. For example, the compositions may provide a therapeutically effective plasma concentration of the therapeutic agent within a period of less than 15, 10, 5, 4, 3 or 2 minutes, or less than 1 minute or 0.5 minutes from application to the skin.

The compositions provided by the present invention achieve the object of sustained transdermal absorption by the use of appropriate delivery kinetics. This is achieved by the use of particular carrier materials in the composition, to alter the hydrophilicity of the composition and thus alter the flux of the active agent from the composition through the stratum corneum. Using this approach, the solvent properties of the composition, and therefore the solubility profile of the active agent, can be modified which means that the rate of diffusion of the active agent through the skin can be controlled.

The compositions for sustained administration of the therapeutic agent may be used in therapy or prophylaxis and, in particular, for treating or preventing pain and/or inflammation, nausea and vomiting, dementia, Parkinson's disease or essential tremor, chorea, tics and related disorders. The present invention is also suitable for use in nicotine-replacement therapy, hormone-replacement therapy or contraception. In some embodiments, the present invention is not used for the treatment of respiratory diseases, pain or inflammation or the administration of local anesthetics.

The therapeutic agent is a substance which has therapeutic or prophylactic effects. Therapeutic agents which may advantageously be included in the compositions include those which are usually administered for the treatment of pain and/or inflammation, nausea and/or vomiting, Parkinson's disease, essential tremor, chorea, tics and related disorders as hereinbefore described. The therapeutic agents may also include those which are usually administered for the treatment of dementia, for example, donepezil hydrochloride, galantamine, memantine hydrochloride, rivastigmine. The therapeutic agent may include those commonly used in hormone-replacement therapy or contraception, for example, testosterone, dydrogesterone, medroxyprogesterone acetate, norethisterone, progesterone, levonorgestrel, conjugated oestrogens, estradiol, estrone, estriol, estropipate. The therapeutic agent may be one used in nicotine replacement therapy, such as nicotine or bupropion hydrochloride, which are used to reduce cravings. The therapeutic agent may be a water-soluble vitamin, for example, Vitamin B1, B2, B3, B6 or B12, Vitamin C, Folic acid or biotin. The therapeutic agent may also be omeprazole.

In some embodiments, the compositions according to the second aspect of the present invention comprise a means of substantially occluding the therapeutic agent from the air following application to the skin. The occlusive means preferably comprises appropriate quantities of a wax, oil or fat included in the carrier material of the compositions. Such compositions comprising a means of occlusion are able to provide the advantages associated with the use of medicinal patches and plasters, without the disadvantages.

In particular, the compositions comprising an occluding means increase hydration of the skin beneath the occlusion, thereby enhancing the absorption of the therapeutic agent from the composition. The occluding means also serves to protect the composition from the environment, ensuring that the composition does not get rubbed off, which may result in incomplete administration of the dose of therapeutic agent provided by the composition.

In an embodiment of the invention, the composition comprises one or more film-forming materials, which are capable of forming a protective layer when the composition is applied to the skin of a patient. These film-forming materials do not rapidly melt upon application to the skin, thereby allowing the therapeutic agent to be absorbed across the skin from beneath the protective layer formed.

The inclusion of a film-forming layer in the composition avoids the need for a plaster, or other form of adhesive covering member. This can be advantageous, as discussed above, in avoiding the difficulties associated with the use of plasters and adhesives. Inclusion of a film-forming layer would be of particular use when the composition comprises a carrier material and/or an active agent which may volatise or evaporate at skin temperatures.

In a further embodiment, the sustained release composition according to the second aspect of the present invention has a substantially solid form at a temperature of about 25° C. or less, and a softening point of not higher than the skin temperature of a mammalian patient. Solid compositions which soften and melt upon contact with the skin of a mammalian patient have already been discussed in detail in the preceding passages.

Such composition can be used for the treatment of conditions where the use of plasters is already known, for example, in hormone- or nicotine-replacement therapy.

In further embodiments of the invention, the compositions are incorporated into medicinal plasters or patches which include a covering layer which covers the composition and is to be affixed to the skin of a patient, preferably by an adhesive which is located around the edge of the covering layer. Such plasters or patches will increase hydration of the skin beneath the covering layer when they are applied to the skin, thereby enhancing absorption of the therapeutic agent from the composition. The covering layer also serves to protect the composition from the environment, ensuring that the composition does not get rubbed off, which may result in incomplete administration of the dose of therapeutic agent provided by the composition.

Plasters according to the present invention avoid several of the disadvantages associated with medicinal plasters known in the art. For example, in plasters according to the present invention, the rate of delivery of the drug to the bloodstream is controlled as a result of the composition itself, and does not, therefore, rely upon the use of rate-controlling adhesive layers, membranes, matrices, or adhesion controlled system, which can be technically costly and require special apparatus to make. Further to this, in such embodiments of the present invention, the purpose of the plaster is merely to hold the composition in contact with the skin, and as such, the plaster is prepared for use by simply locating a desired quantity of a composition, prepared in accordance with the second aspect of the present invention, under a covering with an adhesive edge. Unlike plasters known in the art, no special methods of manufacture, which can again be costly, are required in order to incorporate the composition into a rate-controlling layer. The compositions incorporated in the plasters according to the present invention are preferably in the form of a tablet, and are preferably made on a tabletting machine. This is, again, a very different method of producing plasters from the way in which known medicinal plasters are made.

The incorporation of the compositions of the present invention into such plasters or patches is particularly desirable where the compositions are used to administer therapeutic agents over a prolonged period of time. For example, slow or sustained release of agents such as water-soluble vitamins and hormones may be desired over a period of months.

The adhesive used in the medicinal plasters or patches according to the present invention should be compatible with the therapeutic agent.

In certain embodiments, the medicinal plaster or patch further comprises an absorbent material, which is located on the same side of the covering layer as the pharmaceutical composition. In such embodiments, the pharmaceutical composition melts upon contact with the skin and is absorbed by the absorbent material. This avoids the formation of a molten reservoir or pool of non-solid composition under the covering layer, which might otherwise seep out from the plaster or patch over time. The absorbent material may be any substance suitable for the absorption of the pharmaceutical composition, including, for example, felt, cotton wool or polyurethane foam.

Preferably, the carrier medium constitutes not less than about 60%, more preferably not less than about 80% and even more preferably not less than about 90%, by weight based on the weight of the pharmaceutical composition.

Preferably, the therapeutic agent or agents in compositions according to the present invention are present in the pharmaceutical composition in a therapeutically effective concentration of at least 0.01% by weight based on the weight of the pharmaceutical composition.

Pharmaceutical compositions according to the present invention may further comprise, where appropriate, additional ingredients such as one or more penetration enhancers (which may be surfactants, alcohols, esters, glycols or the like or any other suitable penetration enhancer), humectants, surfactants (which may be cationic, non-ionic, anionic or polymeric), emulsifiers, antioxidants, preservatives, clays, antifoaming agents, spreading agents, emollients, barriers, solubilising agents for the therapeutic agent and the like.

Pharmaceutical compositions according to the present invention may also comprise solvents, such as ethanol, menthol, thymol, eucalyptol, eucalyptus oil, benzyl alcohol, isopropyl alcohol, propylene glycol, methylated spirit, phenol, cyclodextrins, ethyl oleate, eugenol, glycerol, levomenol, monoethanolamine oleate, myristyl alcohol, octyldodecanol, methyl alcohol, coconut oil or silicone oil.

The presence of solvents in compositions according to the present invention aids transdermal administration of the therapeutic agent. The extent to which, and speed with which systemic and local administration of a therapeutic agent from a topically applied composition occurs is associated with the depth and rate of penetration of the therapeutic agent through the skin. The compositions provided by the present invention achieve the object of systemic and, in some embodiments, local transdermal administration by the use of particular carrier materials in the composition in order to alter the hydrophilicity as hereinbefore discussed, and the inclusion of solvents. The presence of solvents in compositions according to the present invention aids solubilization of the drug within the composition. Solvents for use in the present invention are also chosen in accordance with their ability to cross or bridge the stratum corneum, and in particular tight junctions between the corneocytes within the stratum corneum. The presence of solvents in the present invention thus affects the rate of transdermal absorption, and the depth of penetration of the therapeutic agent, by solubilizing the agent, and effecting diffusion of the agent through the stratum corneum. The rate and depth of delivery of the therapeutic agent to the bloodstream and/or local receptors, and therefore the effect of the agent can thus be modified in compositions according to the present invention by the selection and use of particular types and quantities of solvents.

Pharmaceutical compositions according to the present invention may further comprise organoleptic agents to improve the organoleptic properties of the composition. Such agents include almond oil, glycerol, linseed oil, monoethanolamine oleate, grape oil, mace oil, isopropyl myristate, isopropyl palmitate, palm kernel oil, theobroma oil, wool alcohols. The inclusion of organoleptic agents can be used, for example, to enhance the feel of the composition, which can improve patient compliance. In addition, such agents can have a perceived cooling effect, which can provide a positive psychological effect.

Pharmaceutical compositions according to the present invention may further comprise sensory cues, such as anise oil, citronella oil, clove oil, eucalyptol, eucalyptus oil, eugenol, juniper oil, lemon grass oil, lemon oil, tepeneless lemon oil, melaleuca oil, neroli oil, nutmeg oil, olive oil, orange oil, terpeneless orange oil, poppy seed oil, pine oil, rose oil, sage oil, spearmint oil, lavender oil, thyme oil, vanillin.

The inclusion of such cues in the composition can provide the patient with pleasant sensory feedback upon use, allows the patient and/or person applying the formulation to recognize that administration has occurred, and may aid recollection of administration. Such factors can improve patient compliance and provide a positive psychological effect.

Pharmaceutical compositions according to the present invention may further comprise insect repellents such as citronella or lemon grass.

In some embodiments of the present invention, the compositions are substantially free of penetration enhancers. In such embodiments, the compositions are preferably prepared using a process carried out under aseptic conditions.

The use of preservatives can be undesirable, as they may provoke allergic reactions in susceptible patients, and the present invention may be advantageous in avoiding or reducing the risk of such allergic reactions. Preservatives that have been associated with allergic reactions include chlorocresol, hydroxybenzoates (parabens), polysorbates, sorbic acid and the like, and these preservatives are included in a large number of known topical compositions, including, for example, compositions available under any of the following trade marks: Drapolene, Medicaid, Siopel, Sprilon, Eurax, Efcortelan, Mildison, Fucidin H, Nystaform, Quinocort, Terra-Cortril Nystatin, Timodine, Locoid, Locoid Crelo, Modrasone, Propaderm, Betnovate, Betnovate RD, Diprosone, Dermovate, Eumovate, Trimovate, Nerisone, Haelan, Synalar, Ultralanum Plain, Zorac, Carbo-Dome, Exorex, Differin and Exelderm.

In a particularly preferred embodiment of the present invention, the pharmaceutical compositions are substantially free of the types of preservative generally included in compositions intended for dermal or transdermal administration, or at least they include such preservatives in amounts that are less than those generally required in compositions intended for dermal or transdermal administration, or they include such preservatives in amounts that generally do not provoke substantial allergic reactions in susceptible patients, substantially as hereinafter described.

The preservatives generally employed in compositions intended for dermal or transdermal administration are included to prevent or reduce contamination of such compositions. Contamination is a particular problem where a composition is repeatedly exposed to the atmosphere or is repeatedly handled. Preservatives may not be required in compositions of the present invention where the compositions are in the form of unit doses, especially if these doses are individually packaged.

Pharmaceutical compositions according to the present invention may, however, comprise one or more preservatives, such as phenoxyethanol or the like, that are included typically to substantially prevent contamination of the compositions according to the present invention during manufacture but are not generally of the type employed to prevent infection due to manual application as hereinbefore described.

In further embodiments of the present invention, the compositions are substantially free of antioxidants. In such cases, it is preferred that the compositions are packaged in a substantially inert atmosphere, such as nitrogen or the like.

The use of antioxidants can provoke allergic reactions in susceptible patients and the present invention may be advantageous in avoiding or reducing the risk of such allergic reactions in susceptible patients. Antioxidants that have been associated with allergic reactions include butylated hydroxyanisole, butylated hydroxytoluene and the like, and are known to be available in prior art topical compositions, such as those compositions available under any of the trade marks Imuderm, Siopel and the like.

The compositions according to the present invention may be used to provide patients with a combination of rapid and sustained administration of one or more therapeutic agents. It is known, for example, that one reason why some smokers fail to give up smoking is because the sustained levels of nicotine or therapeutic agent provided by patches provided as part of a nicotine-replacement program do not accurately reflect the effects of smoking. In particular, the patches currently available are unable to provide a substitute for the peak nicotine blood levels that are experienced upon smoking a cigarette. For example, nicotine patches typically provide nicotine levels in the blood of 10-15 ng/ml approximately 4-6 hours after adhesion of a medicinal patch to the skin. In comparison, smoking a cigarette provides a blood nicotine peak of about 25 ng/ml approximately 5 minutes after smoking is commenced.

A combination of rapid and sustained administration may also be of benefit to patients with conditions in which it is desirable to maintain blood levels of the therapeutic agent, but also provide a means of rapid administration in order to alleviate worsening of symptoms or specific “episodes”.

Thus, in some embodiments of the present invention, combinations of rapid and sustained drug administration are provided by at least one composition according to the first or second aspect of the present invention. A combination therapy could comprise two or more transdermal compositions as described herein, or a combination of a transdermal composition according to the present invention and a therapeutic agent formulated for administration via another route, for example a sustained release oral dosage form.

According to a third aspect of the present invention, there is provided an applicator for topically applying to the skin or other exterior region of a human or animal body, a pharmaceutical composition according to either the first or second aspects of the present invention, said applicator comprising a receiving means for receiving and carrying the pharmaceutical composition so that the composition may be applied directly to the skin, and a grip for enabling a user to hold and manipulate the applicator.

In a preferred embodiment, the composition is substantially solid at temperatures below 25° C. and softens upon contact with the skin of the patient. The composition may be in the form of a solid unit dosage form.

In alternative embodiments, the applicator may also include an intermediate member attached to the composition, and the receiving means of the applicator may be configured to be removably attachable to the intermediate member.

Preferably, such applicators are provided in individual, sealed packaging.

According to a fourth aspect of the present invention, a kit is provided comprising a pharmaceutical composition according to the first or second aspect of the present application. The kit may further include an applicator, such as an applicator according to the third aspect of the invention.

In one embodiment, the kit comprises a pharmaceutical composition for rapid administration of a therapeutic agent, and a pharmaceutical composition for sustained administration of the same or a different therapeutic agent, at least one of the compositions being in accordance with the first or second aspect of the present invention. The kit may comprise compositions in accordance with both the first and second aspects of the present invention, providing both rapid and sustained administration using compositions of the present invention.

Such a kit would be of importance in conditions where sustained administration of the therapeutic agent is required, but where it is also desirable to rapidly and transiently raise blood levels of the therapeutic agent, as discussed above.

In one embodiment, the kit comprises at least one dose of the pharmaceutical composition for sustained transdermal administration, and more than one dose of the pharmaceutical composition for rapid transdermal administration, for administration at regular intervals throughout the period for which one sustained transdermal composition is intended. The number of doses of the pharmaceutical composition for rapid administration and for sustained administration may be provided in accordance with medical recommendations.

In one embodiment, the kit comprises at least one dose of the pharmaceutical composition for sustained transdermal administration, and a sufficient number of doses of the pharmaceutical composition for rapid transdermal administration to be taken at regular intervals throughout the period for which one sustained transdermal composition is intended, and one or more applicators according to the second aspect of the present invention. Kits in accordance with the present invention can include instructions for use.

Various embodiments of the present invention will now be illustrated by the following Examples, which do not limit the invention in any way.

### EXAMPLE 1

| Ingredients: | % w/w |
|---|---|
| Softisan 142 | 70 |
| Migylol 812 N | 5 |
| Lexol IPM | 2.5 |
| Fitoderm | 1.5 |
| Dry Flo AF Pure | 10 |
| Propylene Glycol | 1 |
| Nicotine | 10 |

### Method of Preparation for Example 1

All ingredients excluding the nicotine and dry flo were melted down until molten, and the temperature of the bulk was then maintained at 60° C. The nicotine and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 2

| Ingredients: | % w/w |
|---|---|
| Softisan 142 | 80 |
| Migylol 812 N | 5 |
| Lexol IPM | 2.5 |
| Fitoderm | 1.5 |
| Dry Flo AF Pure | 10 |
| Propylene Glycol | 1 |
| Estradiol | 0.005 |
| Norethisterone | 0.25 |

### Method of Preparation for Example 2

All ingredients excluding the estradiol, norethisterone and dry flo were melted down until molten, and the temperature of the bulk was then maintained at 60° C. The estradiol, norethisterone and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 3

| Ingredients: | % w/w |
|---|---|
| Softisan 142 | 79 |
| Migylol 812 N | 5 |
| Lexol IPM | 2.5 |
| Fitoderm | 1.5 |
| Dry Flo AF Pure | 10 |
| Propylene Glycol | 1 |
| Hyoscine | 1 |

### EXAMPLE 4

| Ingredients | % w/w |
|---|---|
| Softisan 133 | 80 |
| Migylol 812 N | 5 |
| Lexol IPM | 2.5 |
| Fitoderm | 1.5 |
| Dry Flo AF Pure | 10 |
| Hyoscine | 1 |

### Methods of Preparation for Examples 3 and 4

All ingredients excluding the hyoscine and dry flo were melted down until molten, and the temperature of the bulk was then maintained at 60° C. The hyoscine and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 5

| Ingredients: | % w/w |
|---|---|
| Softisan 142 | 78 |
| Migylol 812 N | 5 |
| Lexol IPM | 2.5 |
| Fitoderm | 1.5 |
| Dry Flo AF Pure | 10 |
| Propylene Glycol | 1 |
| Selegiline | 2 |

### Method of Preparation for Example 5

All ingredients excluding the selegiline and dry flo were melted down until molten, and the temperature of the bulk was then maintained at 60° C. The selegiline and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 6

| Ingredients: | % w/w |
|---|---|
| Softisan 142 | 79 |
| Migylol 812 N | 5 |
| Lexol IPM | 2.5 |
| Fitoderm | 1.5 |
| Dry Flo AF Pure | 10 |
| Propylene Glycol | 1 |
| Galantamine | 1 |

### Method of Preparation for Example 6

All ingredients excluding the galantamine and dry flo were melted down until molten, and the temperature of the bulk was then maintained at 60° C. The galantamine and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 7

| Ingredients: | % w/w |
|---|---|
| Softisan 133 | 80 |
| Migylol 812 N | 5 |
| Lexol IPM | 2.5 |
| Fitoderm | 1.5 |
| Dry Flo AF Pure | 10 |
| BCG Vaccine | 1 dosage |

### Method of Preparation for Example 7

All ingredients excluding the vaccine and dry flo were melted down until molten, and the temperature of the bulk was then maintained at 60° C. The vaccine and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 8

| Ingredients: | % w/w |
|---|---|
| Softisan 142 | 70 |
| Migylol 812 N | 5 |
| Lexol IPM | 2.5 |
| Fitoderm | 1.5 |
| Dry Flo AF Pure | 10 |
| Ethanol | 1 |
| Nicotine | 10 |

### Method of Preparation for Example 8

All ingredients excluding the nicotine, ethanol and dry flo were melted down until molten, and the temperature of the bulk was then maintained at 60° C. The nicotine was mixed with ethanol and then carefully sheared into the bulk with the dry flo using a Silverson mixer. Once the bulk was sufficiently cool (for example, less than 20° C.), the ethanol was mixed in. The bulk was solidified by exposing to low temperature, for example, 4° C. The solidified bulk was milled down and granulated also at low temperature, for example, 4° C.

### EXAMPLE 9

| Ingredients: | % w/w |
|---|---|
| Softisan 142 | 79 |
| Migylol 812 N | 5 |
| Lexol IPM | 2.5 |
| Fitoderm | 1.5 |
| Dry Flo AF Pure | 10 |
| Ethanol | 1 |
| Hyoscine | 1 |

### Method of Preparation for Example 9

All ingredients excluding the hyoscine, ethanol and dry flo were melted down until molten, and the temperature of the bulk was then maintained at 60° C. The hysocine and dry flo were carefully sheared into the bulk using a Silverson mixer. Once the bulk was sufficiently cool (for example, less than 20° C.), the ethanol was mixed in. The bulk was solidified by exposing to low temperature, for example, 4° C. The solidified bulk was milled down and granulated also at low temperature, for example, 4° C.

### EXAMPLE 10

| Ingredients: | % w/w |
|---|---|
| Softisan 142 | 78.745 |
| Migylol 812 N | 5 |
| Lexol IPM | 2.5 |
| Fitoderm | 1.5 |
| Dry Flo AF Pure | 10 |
| Propylene Glycol | 1 |
| Gantrez ES 425 | 1 |
| Estradiol | 0.005 |

Norethisterone 0.25

### Method of Preparation for Example 10

All ingredients excluding the estradiol, norethisterone, gantrez ES 425 and dry flo were melted down until molten, and the temperature of the bulk was then maintained at 60° C. The estradiol, norethisterone and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 11

| Ingredients: | % w/w |
|---|---|
| Softisan 142 | 69 |
| Migylol 812 N | 5 |
| Lexol IPM | 2.5 |
| Fitoderm | 1.5 |
| Dry Flo AF Pure | 10 |
| Propylene Glycol | 1 |
| Gantrez ES 425 | 1 |
| Nicotine | 10 |

### Method of Preparation for Example 11

All ingredients excluding the nicotine, gantrez ES 425 and dry flo were melted down until molten, and the temperature of the bulk was then maintained at 60° C. The nicotine and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 12

| Ingredients: | % w/w |
|---|---|
| Softisan 133 | 83 |
| Dry Flo AF Pure | 10 |
| Migylol 812N | 3.5 |
| Fitoderm (veg squalene) | 1 |
| Glyceryl Trinitrate | 2.5 |

### Method of Preparation for Example 12

All ingredients excluding the glyceryl trinitrate and dry flo were melted down until molten, and the temperature of the bulk was then maintained at 60° C. The glyceryl trinitrate and dry flo were carefully sheared into the bulk using a Silverson mixer. The bulk was solidified by exposing to low temperature, for example, 4° C. The solidified bulk was milled down and granulated also at low temperature, for example, 4° C.

Percentages are by weight based on the total weight of the combined ingredients.

1. A pharmaceutical composition for topical application comprising a therapeutic agent and a pharmaceutically acceptable carrier, for systemic transdermal administration of the therapeutic agent, providing a therapeutic effect within a period of no more than 20 minutes from topical application to the subject.

2. A pharmaceutical composition as claimed in claim 1, wherein the composition provides transdermal administration of the therapeutic agent within a period of no more than 15, 10, 5, 4, 3, 2 minutes, 1 minute or 30 seconds from topical application to a subject.

3. A pharmaceutical composition as claimed in claim 1, wherein the therapeutic agent is a vaccine, or is an agent known for the treatment of nausea, vomiting, Parkinson's disease, essential tremor, chorea or tics, or smoking cessation.

4. A pharmaceutical composition as claimed in claim 1, wherein the carrier is glyceride, cocoa butter, theobroma, a high molecular weight polyethylene glycol, a polyoxyethylene, lanolin or a derivative thereof, a fatty acid, fatty alcohol or fatty ester, or an organic oil.

5. A pharmaceutical composition as claimed in claim 1, wherein the composition is substantially free of preservatives.

6. A pharmaceutical composition as claimed in claim 1, wherein the composition further comprises one or more preservatives to prevent or reduce contamination of the composition during preparation.

7. A pharmaceutical composition, according to claim 1, wherein the composition is substantially free of antioxidants.

8. A pharmaceutical composition according to claim 1, wherein the carrier constitutes not less than about 60% by weight based on the weight of the pharmaceutical composition.

9. A pharmaceutical composition as claimed in claim 1, wherein the composition further comprises one or more solvents.

10. A pharmaceutical composition as claimed in claim 9, wherein the solvent comprises ethanol, menthol, thymol, eucalyptol, eucalyptus oil, benzyl alcohol, isopropyl alcohol, propylene glycol, methylated spirit, phenol, cyclodextrins, ethyl oleate, eugenol, glycerol, levomenol, monoethanolamine oleate, myristyl alcohol, octyldodecanol, methyl alcohol, coconut oil or silicone oil.

11. A pharmaceutical composition as claimed in claim 1, wherein the composition is spreadable at room temperature.

12. A pharmaceutical composition as claimed in claim 11, wherein the composition is in the form of an ointment, cream or gel.

13. A pharmaceutical composition as claimed in claim 1, wherein the composition has a substantially solid form at a temperature of about 25° C. or less and a softening point of not higher than the skin temperature of a mammalian patient.

14. A pharmaceutical composition as claimed in claim 13, wherein the composition is provided as a solid tablet.

15. A pharmaceutical composition as claimed in claim 1, further comprising a means of substantially occluding the pharmaceutical composition from the air upon application.

16. A pharmaceutical composition as claimed in claim 15, further comprising a film-forming material ingredient as an occlusive means.

17. A pharmaceutical composition as claimed in claim 15, wherein the carrier material includes a wax, oil or fat as the occluding means.

18. A pharmaceutical composition as claimed in claim 1, wherein the composition provides systemic transdermal administration of the therapeutic agent over a period of at least 30 minutes from topical application to the subject.

19. A pharmaceutical composition as claimed in claim 17, wherein the composition provides transdermal administration of the therapeutic agent over a period of at least about 1, 2, 4, 8, 12, 16, 20, 24, 26, 28, 32, 36, 48 or 72 hours.

20. A pharmaceutical composition as claimed in claim 19, wherein the therapeutic agent is an agent used in nicotine-replacement therapy or hormone-replacement therapy, or is used to provide contraception or is a water-soluble vitamin.

21. A pharmaceutical composition as claimed in claim 20, wherein the therapeutic agent is nicotine, bupropion hydrochloride, testosterone, dydrogesterone, medroxyprogesterone acetate, norethisterone, progesterone, levonorgestrel, conjugated oestrogens, estradiol, estrone, estriol or estropipate.

22. A medicinal plaster comprising a pharmaceutical composition as claimed in claim 1.

23. An applicator for applying a pharmaceutical composition as claimed in claim 1.

24. Use of a therapeutic agent in the manufacture of a medicament comprising a composition as claimed in claim 1.

25. A use as claimed in claim 24, wherein the medicament is for treating nausea, vomiting, dementia, Parkinson's disease, essential tremor, chorea or tics, or for providing hormone- or nicotine-replacement therapy or for administering vitamins, contraception, or a vaccine.

26. A pharmaceutical composition or product substantially as described in any one of the Examples.
